(19) United States
(12) Patent Application Publication
(10) Pub. No.: US 2023/0190631 A1
(43) Pub. Date: Jun. 22, 2023

(54) SKIN CARE COMPOSITIONS

(71) Applicants: Mersedeh OSSUDALLAH, North Vancouver (CA); Goltan BERYANI, North Vancouver (CA)

(72) Inventors: Mersedeh OSSUDALLAH, North Vancouver (CA); Goltan BERYANI, North Vancouver (CA)

(21) Appl. No.: 18/085,312

(22) Filed: Dec. 20, 2022

Related U.S. Application Data
(60) Provisional application No. 63/291,664, filed on Dec. 20, 2021.

(51) Int. Cl. A61K 8/92; A61K 8/31; A61K 8/9794; A61K 8/49; A61Q 19/00; A61Q 19/08

(52) U.S. Cl. CPC A61K 8/922; A61K 8/31; A61K 8/9794; A61K 8/498; A61Q 19/007; A61Q 19/08

(57) ABSTRACT

A skin care composition for improving the appearance of the skin. The skin care composition comprises petroleum jelly, a vegetable oil blend and an antioxidant. The concentration of the vegetable oil blend in the composition is at least 50% (v/v). In some embodiments, the vegetable oil blend comprises apricot oil, coconut oil, jojoba oil and argan oil. In some embodiments, the vegetable oil blend further comprises castor oil and vegetable glycerin oil. The skin care composition may optionally further include an essential oil such as lavender oil and a substance from the plant of the Aloe genus.

### FIELD OF THE INVENTION

The invention pertains to cosmetic compositions, in particular to skin care compositions for topical application to the skin.

### BACKGROUND OF THE INVENTION

Many ingredients are known to be used in skin care preparations. Such ingredients may be natural ingredients (i.e., plant, animal or microbial ingredients) such as plant oil, green tea and the like, or chemically synthesized ingredients, such as parabens, formaldehyde and the like. The selection of the types of ingredients, and the ratios and/or concentrations of which to include in the composition can have an impact on the effectiveness of the product. The effectiveness of a skin care product relates to the effect on the appearance of the skin of users after application of the composition. This present invention is directed to skin care compositions that can improve the skin appearance on users of different skin types.

### SUMMARY

An aspect of the invention provides a topical skin care composition that may be formulated as a face serum which has been shown to improve the appearance of the skin of users with different skin types. The skin care composition comprises petrolatum, a vegetable oil blend, an antioxidant, and optionally an essential oil and a substance obtained from a plant of the Aloe genus. The vegetable oil blend may include a combination of apricot oil, coconut oil, jojoba oil, argan oil, and optionally castor oil and/or vegetable glycerin oil. In some embodiments, the vegetable oil blend contained in the composition is at least 50% (v/v), or at least 60% (v/v).

In an example embodiment, the skin care composition comprises: about 5% to 9% (v/v) of petroleum jelly; about 60% to about 70% (v/v) of a vegetable oil blend, wherein the vegetable blend comprises about 5% to about 9% (v/v) of apricot oil, 5% to about 9% (v/v) of coconut oil, about 5% to about 9% (v/v) of joboba oil, about 20% to about 35% (v/v) of argan oil, about 5% to about 9% (v/v) of vegetable glycerin oil, and about 5% to about 9% (v/v) of castor oil; about 5% to about 9% (v/v) of vitamin E; about 5% to about 9% (v/v) lavender oil; and about 5% to about 9% (v/v) of aloe vera.

In an example embodiment, the skin care composition consists essentially of: about 5% to 9% (v/v) of petroleum jelly; about 60% to about 70% (v/v) of a vegetable oil blend, wherein the vegetable blend comprises about 5% to about 9% (v/v) of apricot oil, 5% to about 9% (v/v) of coconut oil, about 5% to about 9% (v/v) of joboba oil, about 20% to about 35% (v/v) of argan oil, about 5% to about 9% (v/v) of vegetable glycerin oil, and about 5% to about 9% (v/v) of castor oil; about 5% to about 9% (v/v) of vitamin E; about 5% to about 9% (v/v) lavender oil; and about 5% to about 9% (v/v) of aloe vera.

Further aspects of the invention and features of specific embodiments of the invention are described below.

### DETAILED DESCRIPTION

The invention pertains to topical skin care compositions which have conditioning and moisturizing properties. As used herein, “conditioning” relates to the deposition of an emollient on the skin, which results in the delay in moisture loss from the skin, thereby maintaining and restoring the softness and smoothness of the skin surface. A product “moisturizes” when water which is normally lost to the atmosphere is accumulated in the stratum corneum, the outermost layer of the skin. “Moisturizing” prevents and alleviates skin irritation, and soothes the skin by slowing the evaporation of water. The skin care composition also provides cosmetically acceptable tactile properties, i.e., composition does not leave the skin too greasy when applied thereon. As an example, petrolatum or petroleum jelly, when applied to the skin alone, may be considered by some users to be too greasy and thus cosmetically unacceptable.

In example embodiments, the skin care composition of this invention is formulated in the form of a face serum. Face serums contain high concentrations of ingredients. Users have reported that their skin felt smooth and hydrated after application of the skin care compositions of this invention. The skin care compositions of this invention have been shown to improve the appearance of the skin of users with different skin types, including normal, dry, oily, combination (both oily and dry skin) and sensitive.

In some embodiments, the skin care composition comprises organic ingredients. In some embodiments, the skin care composition comprises more than about 80% wt. of organic ingredients. In some embodiments, the skin care composition comprises more than about 90% wt. of organic ingredients. “Organic ingredients” may be referred to as ingredients containing chemical compositions having carbon atoms. In some embodiments, “organic ingredients” are ingredients that are derived from plants or animals. In some embodiments, the skin care composition is free from niacinamide, hyaluronic acid, glycolic acid, retinol, derivatives thereof, and/or compounds related thereto. In some embodiments, the skin care composition is free from ingredients with known allergenic potential, such as perfumes and/or fragrances, preservatives, colorings, and the like.

In some embodiments, the skin care composition comprises petrolatum, a vegetable oil blend, and an antioxidant. Petrolatum, also known as petroleum jelly, may be a product sold under the trademark Vaseline™. In some embodiments, the skin care composition consists essentially of petroleum jelly, the vegetable oil blend and the antioxidant. In example embodiments, the concentration ratio of petroleum jelly to vegetable oil blend to antioxidant in the composition may be in the range of from about 1:4:1 to about 1:8:1. In other example embodiments, the concentration ratio of petroleum jelly to vegetable oil blend to antioxidant in the composition is approximately 1:5.5:1. In further example embodiments, the concentration ratio of petroleum jelly to vegetable oil blend to antioxidant in the composition is approximately 1:7.5:1.

The vegetable oil blend includes more than one type of vegetable oil. As used herein, the term “vegetable oil” refers to any oil that comes from a plant source. Non-limiting examples of vegetable oils that can be used to produce the vegetable oil blend include coconut oil, castor oil, apricot oil, jojoba oil, argan oil, almond oil, avocado oil, borage oil, camellia tea oil, evening primose oil, grapeseed oil, hemp seed oil, macademia oil, olive oil, peach kernel oil, peanut oil, pistachio oil, neem oil, rosehip oil, sesame oil, palm oil, soybean oil, cottonseed oil, and safflower oil.

In some embodiments, the concentration (v/v) of vegetable oil blend in the composition is more than 50%. In some embodiments, the concentration (v/v) of vegetable oil blend in the composition is about 50% to about 80%.

In example embodiments, the vegetable oil blend comprises apricot oil, coconut oil, jojoba oil and argan oil. The vegetable oil blend may comprise approximately equal amounts of apricot oil, coconut oil and jojoba oil. In some embodiments, the concentration (v/v) of argan oil in the vegetable oil blend is about 20% (v/v) to about 60% (v/v). In some embodiments, the concentration (v/v) of argan oil in the vegetable oil blend is about 30% (v/v) to about 40% (v/v). In some embodiments, argan oil is in an amount greater than the amount of each of apricot oil, coconut oil, jojoba oil contained in the vegetable oil blend. In example embodiments, the concentration ratio of apricot oil, coconut oil, jojoba oil and argan oil in the vegetable oil blend is about 1:1:1:2, or 1:1:1:2.5, or 1:1:1:3. In some embodiments, the vegetable oil blend consists essentially of apricot oil, coconut oil, jojoba oil and argan oil. In other embodiments, the vegetable oil blend comprises castor oil. The amount of castor oil contained in the vegetable oil blend may be appropriately equal to each of the amount of apricot oil, coconut oil and jojoba oil contained in the vegetable oil blend. In further embodiments, the vegetable oil blend comprises vegetable glycerin (or glycerol). The amount of vegetable glycerin contained in the vegetable oil blend may be appropriately equal to each of the amounts of apricot oil, coconut oil and jojoba oil contained in the vegetable oil blend.

As used herein, the term “antioxidant” refers to any substance or mixture of subtances that inhibit or prevent oxidation of cells. Non-limiting examples of antioxidants include vitamin A, vitamn C, vitamn B, vitamn E, coenzyme Q10 or coQ10, polyphenols, and derivatives thereof. In an example embodiment, the antioxidant is vitamin E8.

In some embodiments, the concentration (v/v) of each of petroleum jelly and the antioxidant in the composition is in the range of from about 1% to 20%. In some embodiments, the concentration (v/v) of each of petroleum jelly and the antioxidant in the composition is in the range of from about 5% to 15%.

In some embodiments of the composition, the composition comprises petroleum jelly, a vegetable oil blend, an antioxidant and one or more other ingredients that improve the appearance of the skin. The one or more other ingredients may be natural ingredients. As used herein, “natural ingredients” include plant, animal or microbial ingredients. The natural ingredients contained in the composition are preferably substances that are obtained from plants.

In some embodiments, the composition optionally comprises an essential oil or a blend of essential oils. As used herein, an “essential oil” refers to one or more liquids extracted from plants. Essential oils may be made by steaming or pressing parts of a plant such as flowers, bark, leaves or fruit to capture the desired compound(s). Non-limiting examples of suitable essential oils that can be included in this composition include one or more of lavender oil, tea tree oil, peppermint oil, lemon oil, oregano oil, cinnamon bark oil, jasmine oil, lemongrass oil, chamomile oil, bergamot oil, orange oil, and marjoram oil. In an example embodiment, the essential oil comprises lavender oil.

In some embodiments, the concentration (v/v) of the essential oil or the blend of essential oils contained in the composition is in the range of from about 1% to 20%. In some embodiments, the concentration (v/v) of the essential oil or the blend of essential oils contained in the composition is in the range of from about 5% to 15%.

In some embodiments, the composition optionally comprises a substance obtained from a plant of the Aloe genus. In some embodiments, the plant is Aloe vera, and the substance is a liquid obtained therefrom.

In some embodiments, the concentration (v/v) of the substance obtained from the plant of the Aloe genus contained in the composition is in the range of from about 1% to 20%. In some embodiments, the concentration (v/v) of the substance obtained from the plant of the Aloe genus contained in the composition is in the range of from about 5% to 15%.

The specific types of ingredients to include in the composition (e.g., the specific types of vegetable oils to include in the vegetable oil blend, the specific type of antioxidant, or the types of the one or more other ingredients to include) may be selected based on the specific benefits that the composition intends to provide. For example, the ingredients may be selected based on the specific skin problem to be targeted. The specific skin problem may for example be one or more of correcting the evenness of skin tone and texture, improving the uniformity of colour, improving skin elasticity, reducing the visibility of fine lines and wrinkles, reducing dark spots, and improving hydration. For example, some compositions may be targeted to users with dry and/or dull skin. For such users, the composition may include ingredients that provide a desired amount of hydration to the skin. Such ingredients may include for example one or more of vegetable glycerin, coconut oil and aloe vera. In another example, some compositions may be targeted to users with undesired fine lines and wrinkles. For such users, the composition may include ingredients that facilitate the reduction of fine lines and wrinkles. Such ingredients may include for example one or more of vitamin A and vitamin C compounds and coenzyme Q10.

In some embodiments, the petrolatum or petroleum jelly is substituted substantially or partially with a petroleum jelly replacement, i.e., a composition having properties similar to or substantially similar to petroleum jelly without one or more ingredients contained therein, or with one or more ingredients being substituted by another one or more other ingredients. In some embodiments, the petroleum jelly replacement comprises vegetable oil and silica (such as fumed silica powder). The petroleum jelly replacement may also comprise other ingreidents such as vitamin E, margarine or butter and/or starch, etc. Other suitable composition(s) to replace petrolatum or petroleum jelly may be used.

The skin care composition of the present invention may be applied once or twice daily, in the morning and/or evening. In some embodiments, the skin care composition is used in the absence of other accompanying skin care products such as eye cream and/or face moisturizers. The skin care composition may be an all-in-one product which serves as one or more of eye cream and/or face moisturizers and/or face lotions and/or face serum. The skin care composition may be applied to all parts of a user's face, including without limitation, forehead, cheeks, nose, and sensitive areas such as under-eye, eyelid, and chin.

Some aspects of the invention pertain to use of the skin care composition. The use of the skin care composition may achieve one or more of the following desirable results:
clears and/or brightens the skin;
removes discoloration and/or scarring such as acne scars;
reduces or eliminates under-eye bags or dark circles;
reduces dryness such as from eczema;
soothes sensitive skin; and
improves overall complexion.

In some embodiments, use of the skin care composition comprises applying the composition on the user's skin (e.g., on his or her face and/or neck) at least once per day. In some embodiments, each application of the skin care composition comprises applying about 0.05 to about 0.5 mL (equivalent to about 1 to 10 drops) of the composition, or in some embodiments, applying about 0.10 to about 0.25 mL (equivalent to about 2 to 5 drops) of the composition, or in some embodiments, applying about 0.15 to about 0.20 mL (equivalent to about 3 to 4 drops) of the composition.

### Example

A skin care formulation according to the invention is prepared in accordance with the following composition:

| | | |
|---|---|---|
| VASELINE ™ | 1 Tsp | 8.70% (v/v) |
| CASTOR OIL | 1 Tsp | 8.70% (v/v) |
| APRICOT OIL | 1 Tsp | 8.70% (v/v) |
| LAVENDER OIL | 1 Tsp | 8.70% (v/v) |
| COCONUT OIL | 1 Tsp | 8.70% (v/v) |
| JOJOBA OIL | 1 Tsp | 8.70% (v/v) |
| ALOE VERA | 1 Tsp | 8.70% (v/v) |
| VEGETABLE GLYCERIN OIL | 1 Tsp | 8.70% (v/v) |
| ARGAN OIL | 2.5 Tsp | 21.74% (v/v) |
| VITAMIN E8 | 1 Tsp | 8.70% (v/v) |

This formulation was applied to the skin. After a week of daily application both in the morning and afternoon, users have observed one or more of: softened and rehydrated skin, a reduction of dark spots, and absent of any allergic responses or other undesired responses such as skin irritation, e.g., redness or swollenness.

Throughout the foregoing description and the drawings, in which corresponding and like parts are identified by the same reference characters, specific details have been set forth in order to provide a more thorough understanding to persons skilled in the art. However, well known elements may not have been shown or described in detail or at all to avoid unnecessarily obscuring the disclosure.

As will be apparent to those skilled in the art in the light of the foregoing disclosure, many alterations and modifications are possible in the practice of this invention without departing from the scope thereof. Accordingly, the description and drawings are to be regarded in an illustrative, rather than a restrictive, sense.

1. A skin care composition comprising petroleum jelly, a vegetable oil blend and an antioxidant, wherein the vegetable oil blend contained in the composition has a concentration of greater than about 50% (v/v).

2. The skin care composition according to claim 1, wherein the concentration of the vegetable oil concentration is greater than about 60% (v/v).

3. The skin care composition according to claim 1, wherein the vegetable oil blend comprises a mixture of apricot oil, coconut oil, jojoba oil and argan oil.

4. The skin care composition according to claim 3, wherein the concentration ratio of apricot oil to coconut oil to jojoba oil to argan oil in the vegetable oil blend is 1:1:1:2.5.

5. The skin care composition according to claim 1, wherein the vegetable oil blend further comprises castor oil.

6. The skin care composition according to claim 5, wherein the vegetable oil blend further comprises vegetable glycerin oil.

7. The skin care composition according to claim 1, wherein the ratio of petroleum jelly to the vegetable oil blend to the antioxidant in the composition is 1:7.5:1.

8. The skin care composition according to claim 1, wherein the concentration of the petroleum jelly in the composition is between 5% (v/v) and 15% (v/v).

9. The skin care composition according to claim 1, wherein the concentration of the antioxidant in the composition is between 5% (v/v) and 15% (v/v).

10. The skin care composition according to claim 1, further comprising an essential oil.

11. The skin care composition according to claim 10, wherein the concentration of the essential oil in the composition is between 5% (v/v) and 15% (v/v).

12. The skin care composition according to claim 1, further comprising a substance obtained from a plant of the Aloe genus.

13. The skin care composition according to claim 12, wherein the concentration of the substance obtained from the plant of the Aloe genus is between 5% (v/v) and 15% (v/v).

14. A skin care composition comprising:
petroleum jelly;
a vegetable oil blend, wherein the vegetable blend comprises apricot oil, coconut oil, joboba oil, and argan oil; and
an antioxidant.

15. The skin care composition according to claim 14, wherein the vegetable oil blend further comprises castor oil and vegetable glycerin oil.

16. The skin care composition according to claim 15, wherein the vegetable oil blend consists essentially of apricot oil, coconut oil, joboba oil, argan oil, castor oil and vegetable glycerin oil.

17. The skin care composition according to claim 14, further comprising an essential oil, wherein the essential oil comprises lavender oil.

18. The skin care composition according to claim 14, further comprising a substance obtained from the plant of the Aloe genus.

19. The skin care composition according to claim 14, wherein the skin care composition consists essentially of petroleum jelly, the vegetable oil blend, the antioxidant, an essential oil, and a substance obtained from the plant of the Aloe genus.

20. A skin care composition comprising:
about 5% to about 9% (v/v) of petroleum jelly;
about 60% to about 70% (v/v) of a vegetable oil blend, wherein the vegetable blend comprises about 5% to about 9% (v/v) of apricot oil, about 5% to about 9% (v/v) of coconut oil, about 5% to about 9% (v/v) of joboba oil, about 20% to about 35% (v/v) of argan oil, about 5% to about 9% (v/v) of vegetable glycerin oil, and about 5% to about 9% (v/v) of castor oil;
about 5% to about 9% (v/v) an antioxidant, wherein the antioxidant comprises vitamin E;
about 5% to about 9% (v/v) of an essential oil, wherein the essential oil comprises lavender oil; and
about 5% to about 9% (v/v) of a substance obtained from the plant of the Aloe genus.
